(19) United States
(12) Patent Application Publication
(10) Pub. No.: US 2026/0033947 A1
(43) Pub. Date: Feb. 5, 2026

(54) PROSTHETIC HEART VALVES AND METHODS FOR USE

(71) Applicant: THE BOARD OF TRUSTEES OF THE LELAND STANFORD JUNIOR UNIVERSITY, Stanford, CA (US)

(72) Inventors: Yuanjia Zhu, Stanford, CA (US); Y. Joseph Woo, Stanford, CA (US)

(21) Appl. No.: 19/352,334

(22) Filed: Oct. 7, 2025

Related U.S. Application Data
(63) Continuation of application No. PCT/US24/24525, filed on Apr. 13, 2024.
(60) Provisional application No. 63/459,220, filed on Apr. 13, 2023.

(51) Int. Cl. A61F 2/24; A61L 27/18; A61L 27/36

(52) U.S. Cl. CPC A61F 2/2418; A61L 27/18; A61L 27/3604; A61F 2220/0008; A61F 2220/0075; A61L 2430/20

(57) ABSTRACT

Prosthetic valves are provided, e.g., for replacing a mitral or aortic valve of a patient, that includes an annular valve body including an inlet end, an outlet end, and one or more valve members for selectively opening and closing a passage between the inlet and outlet ends, a sewing cuff attached around an outer perimeter of the valve body, and a flexible skirt removably attached around the outer perimeter adjacent the sewing cuff. The prosthetic valve may be introduced into a patient's body, positioned adjacent a valve annulus, and the sewing cuff may be secured around a perimeter of the valve annulus. The skirt may be secured to tissue adjacent the valve annulus to further seal the prosthetic valve around the valve annulus, or the skirt may be removed from the valve body, e.g., if the annulus is sufficiently sealed by the sewing cuff.

### RELATED APPLICATION DATA

The present application is a continuation of co-pending International Application No. PCT/US2024/024525, filed Apr. 13, 2024, which claims benefit of U.S. provisional application Ser. No. 63/459,220, filed Apr. 13, 2023, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly, to prosthetic heart valves, e.g., for mitral or aortic valve replacement in patients with severe mitral annular or aortic valve calcification, and to systems and methods for using such valves.

### BACKGROUND

Patients with severe mitral annular calcification (“MAC”) often require mitral valve replacement. Severe calcification can complicate mitral surgery due to potential injury to surrounding structures during debridement, impediment of suture placement through the calcium, and a suboptimal size of prosthesis placed inside the MAC. Excessive de-calcification can result in catastrophic consequences such as atrioventricular groove rupture. The residual MAC can make prosthetic valve seating difficult with issues with hemostasis, resulting in paravalvular regurgitations, a significant risk factor for poor patient outcomes and survival. Similar paravalvular leaks are also seen for aortic valve prosthesis implantation when there is significant amount of residual aortic calcification. Surgeons have tried multiple techniques to buttress or seal the gaps using native tissue, prosthetic graft material, or pericardial patch between annular calcium and the sewing cuff of prosthetic valves. These techniques can be extremely challenging and time consuming to perform, sometimes without satisfactory outcomes. The significantly prolonged aortic cross-clamp time and cardiopulmonary bypass times due to the complexity of the case are known to be associated with postoperative morbidity and mortality.

Accordingly, prosthetic valve devices that allows for extra hemostasis suture to minimize paravalvular leaks, e.g., in patients with severe MAC or aortic valve calcification, would be useful. Such prosthetic valve devices should not impede the primary hemostasis implantation sutures to attach the prosthetic valves to the annulus or the desired positions in situ, and should also be easily reversed back to the current standard prosthetic valve designs to be used in patients without valvular calcification.

### SUMMARY

The present application is directed to medical devices and, more particularly, to prosthetic heart valves, e.g., for mitral or aortic valve replacement in patients with severe MAC or valvular calcifications, and to systems methods for using such valves.

In one example, an innovative prosthetic valve device is provided that may solve one or more of the above-mentioned problems, e.g., including a large skirt proximal (closer to the surgeon) to a sewing cuff. For example, for a valve intended for implantation in a mitral valve annulus, the skirt may be closer to an inlet end of the valve than the sewing cuff, while a valve intended for implantation in an aortic valve annulus, the skirt may be closer to an outlet end of the valve than the sewing cuff, e.g., such that the skirt is closer to the surgeon when the annulus is viewed from above, which may facilitate manipulating, securing, and/or removing the skirt, as desired during a procedure.

The skirt allows for an additional layer of graft material to be sewn around a valve annulus to which the valve is implanted, e.g., onto left atrial tissue around a mitral valve annulus, to ensure a perfect seal or hemostasis suture line to eliminate any potential paravalvular leaks. The skirt may completely surround the perimeter of the valve or may only extend partially around the perimeter, if desired, e.g., less than three hundred sixty degrees (360°) around the perimeter. In addition, the width or outer diameter of the skirt may be varied as needed, e.g., having a radial width between about five and twenty millimeters (5.0-20.0 mm). The skirt geometry and thickness may also be varied as needed. The skirt may be designed in a way such that it may be detachable for patients who do not have severe MAC, valvular calcification, and/or otherwise do not need a second layer of anastomosis suture. The skirt may also be provided in a collapsed geometry, e.g., to minimize obstruction of the sewing cuff and/or anatomical features of the patient and/or allow for sewing ring implantation first, followed by skirt expansion, and lastly a second anastomosis suture line, if desired.

In accordance with one example, a prosthetic valve is provided that includes an annular valve body including an inlet end, an outlet end, and one or more valve members for selectively opening and closing a passage between the inlet and outlet ends, a sewing cuff attached around an outer perimeter of the valve body, and a flexible skirt removably attached around the outer perimeter adjacent the sewing cuff. During use, the prosthetic valve may be introduced into a patient's body, positioned adjacent a valve annulus, and the sewing cuff may be secured around a perimeter of the valve annulus. The skirt may be secured to tissue adjacent the valve annulus to further seal the prosthetic valve around the valve annulus, or the skirt may be removed from the valve body, e.g., if the annulus is sufficiently sealed by the sewing cuff. For example, the skirt may be secured around the valve body by one or more sutures that may be cut or otherwise released, e.g., such that the suture(s) may be removed to release the skirt, which may then also be removed.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show an exemplary prosthetic heart valve including a flexible skirt extending diagonally outwardly from a valve body, e.g., having a frustoconical shape with the skirt closer to the inlet of the valve. In the example shown, the positional relationship between the flexible skirt to the sewing cuff may be configured for implantation in an aortic valve position, e.g., with the skirt closer to the outlet of the valve.

FIGS. 2A and 2B show another exemplary prosthetic heart valve including a flexible skirt extending horizontally outwardly from a valve body, e.g., generally within a plane. In the example shown, the positional relationship between the flexible skirt to the sewing cuff may be configured for implantation in a mitral valve position.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

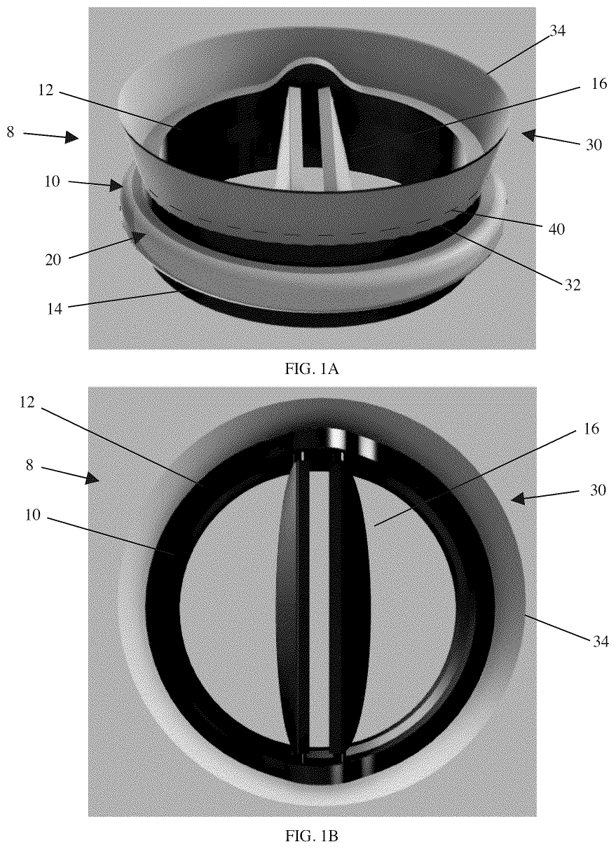

Turning to the drawings, FIGS. 1A and 1B show an example of a prosthetic heart valve 8 that generally includes a valve body 10, a sewing cuff 20, and a skirt 30. As shown, the annular valve body 10 is an enclosed annular structure including an inlet end 12, an outlet end 14, and one or more valve members 16 for selectively opening and closing a passage 18 between the inlet and outlet ends 12, 14. In the example shown, the valve is a mechanical valve including a pair of flap valve members pivotally mounted to the valve body 10, e.g., formed from biocompatible materials, such as plastic, metal, carbon, composite materials, and the like. Alternatively, the valve may be a xenograft, e.g., including a tissue annulus carrying tissue leaflets similar to the valve of the native annulus (not shown), a bioprosthetic valve including leaflets constructed from pericardium or other tissue, or other prosthetic valve.

As shown, the valve body 10 has a substantially circular shape between the inlet and outlet ends 12, 14, although alternatively, the valve body 10 may have other shapes, e.g., a generally “D” cross-sectional shape, a multiple lobed or scalloped outer perimeter (not shown), and the like, for example, similar to the shape of the native annulus into which the prosthetic valve is to be implanted. Although the valve body 10 is shown to lie within a plane, alternatively, the valve body may have a three-dimensional perimeter above or below the plane, e.g., to accommodate the shape of the intended valve annulus. In addition or alternatively, the valve body 10 may have a substantially uniform diameter or shape between the inlet and outlet ends 12, 14, as shown, or may have a tapered or other variable shape between the inlet and outlet ends.

As shown, the sewing cuff 20 is attached around an outer perimeter of the valve body 10, e.g., formed from materials, e.g., fabric, such as polyester (e.g., Dacron) and/or other biocompatible material. The sewing cuff 20 may include a single layer or multiple layers of fabric and/or may be filled with foam, fabric, and/or other material, which may be expandable or non-expandable, to facilitate securing the sewing cuff 20 within or adjacent a native valve annulus (not shown). The sewing cuff 20 may be permanently attached to the valve body 10, e.g., by one or more of bonding with adhesive, fusing, interference fit, one or more connectors, and the like. In the example shown, the sewing cuff 20 may be attached at an intermediate location around the valve body 10 between the inlet and outlet ends 12, 14. Alternatively, the sewing cuff 20 may be attached closer to or immediately adjacent one of the inlet and outlet ends.

In addition, the flexible skirt 30 is attached around the outer perimeter of the valve body 10 adjacent the sewing cuff 20. However, the skirt 30 may be attached around the valve body 10 such that the skirt 30 may be removable from the valve body 10, if desired. For example, an inner edge of the skirt 30 may be secured around the valve body 10, e.g., using one or more sutures (not shown) that may be threaded into or otherwise attached to the skirt material. The suture(s) may be cut and/or untied to allow removal of the skirt from the valve body.

For example, as shown in FIG. 1A, a suture 40 may be provided that may be threaded one or more times in and out of the skirt material around the perimeter of the skirt 30 and whose ends are tied or otherwise secured together, e.g., after sinching or otherwise tightening the suture 40 around the valve body 10. The suture(s) 40 may be formed from conventional materials, such as PTFE and/or other biocompatible materials. To allow the skirt 30 to be removed, the suture 40 may be cut or untied, and a loose end of the suture 40 may be pulled to unthread the suture 40 out of the skirt 30. With the suture(s) removed, the skirt 30 may simply be slipped off from around the valve body 10 and removed.

The skirt 30 may be formed from flexible, biocompatible material, e.g., fabric, such as polyester (e.g., Dacron) and/or other biocompatible materials. For example, the skirt 30 may include one or more layers of fabric extending radially outwardly from the inner edge of the skirt 30 to an outer edge, e.g., having a larger outer diameter or perimeter than the sewing cuff 20. As best seen in FIGS. 1A, the skirt 30 may extend diagonally outwardly from the valve body 10, e.g., to define a generally frusto-conical shape extending outwardly around the inlet end 12 of the valve body 10. For example, the skirt 30 may extend upwardly from the inlet end 12 away from the sewing cuff 20 to provide an additional scaling structure that may be sutured or otherwise secured to tissue surrounding a native valve annulus, as described further elsewhere herein. The configuration shown in FIGS. 1A and 1B may be particularly suited for implantation within a mitral valve annulus, e.g., such that the skirt 30 may be positioned above or otherwise adjacent the inlet of the valve annulus and can be observed and/or manipulated from above the valve annulus. Alternatively, the skirt 30 may be attached adjacent the outlet end of the valve body, e.g., for implantation within an aortic valve annulus.

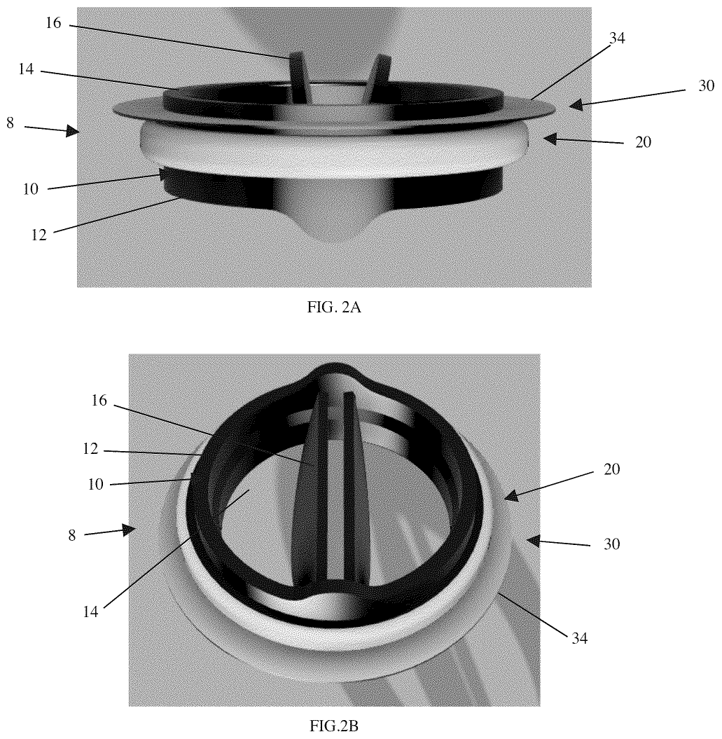

Alternatively, as shown in FIGS. 2A and 2B, a skirt 30 may be attached to a valve body 10 that may extend radially outwardly substantially perpendicular to the valve body 10, e.g., such the skirt 30 is substantially flat. In the example shown in FIGS. 2A and 2B, the skirt 30 is attached adjacent the outlet end 14 of the valve body 10, e.g., such that the skirt 30 may be positioned above an aortic valve annulus when the valve body 10 is positioned within the native valve annulus, e.g., within the aortic root. Alternatively, the skirt 30 may be attached adjacent the inlet end of the valve body, if desired, e.g., for mitral valve implantation. In a further alternative, the skirt may have a three-dimensional shape, e.g., having a non-circular outer perimeter and/or having inner and outer edges that are spaced from the ends of the valve body at different distances around the perimeter.

The skirt 30 may be formed from flexible material to allow the skirt 30 to be manipulated during an implantation material, e.g., to allow the skirt 30 to be directed out of the way to facilitate visualization of the sewing cuff 20, e.g., during positioning and/or suturing the sewing cuff 20 to a native valve annulus. In one example, the skirt 30 may be provided in a collapsed configuration, e.g., rolled, folded, or otherwise positioned close to the valve body 10 to minimize interference with visualizing and/or securing the sewing cuff 20. The skirt 30 may then be expanded, e.g., unrolled, unfolded, and the like, e.g., to contact tissue adjacent the native valve annulus to allow the skirt 30 to be secured to tissue adjacent the native valve annulus.

The valve body 10 and sewing cuff 20 may be sized and/or shaped to be received within or adjacent a native valve annulus, e.g., such that the sewing cuff 20 may be secured to the native annulus using sutures and/or other fasteners. For example, the valve body 10 may be sized to position the sewing cuff 20 within the native annulus such that the inlet and outlet ends 12, 14 are positioned within the heart chambers on either side of the native annulus. In the example shown in FIGS. 1A-IC, the valve body 10 is configured for implantation within or adjacent a mitral valve annulus, e.g., with the skirt 30 closer to the inlet end 12 of the valve body 10, i.e., proximal to the sewing cuff 20. Alternatively, in the example shown in FIGS. 2A-2C, the valve body may be configured for implantation within or adjacent an aortic valve annulus, e.g., with the skirt 30 closer to the outlet end of the valve body.

During use, the prosthetic valve 8 may be introduced into a patient’ heart and positioned adjacent a native valve annulus, e.g., using conventional open or minimally invasive procedures. For example, the heart may be surgically opened to expose the native annulus, which may be prepared, e.g., by removing native leaflets (or a previous prosthetic valve) and/or removing existing calcifications. In the example shown in FIGS. 1A and 1B, the valve 8 may be configured for implantation in a mitral valve annulus, while, in the example shown in FIGS. 2A and 2B, the valve 8 may be configured for implantation in an aortic valve annulus. It will be appreciated that the valve may be otherwise configured for implantation within a tricuspid or pulmonic valve annulus, if desired.

For example, with the valve 8 shown in FIGS. 1A-IC, the valve body 10 may be positioned in the native annulus of the mitral valve, e.g., to dilate or otherwise support the surrounding tissue with the inlet end 12 adjacent the left atrium and the outlet end 14 adjacent the left ventricle (not shown). Thus, the sewing cuff 20 may be positioned within the native annulus and then secured using conventional methods, e.g., threading one or more sutures through the sewing cuff 20 and adjacent tissue.

Alternatively, the valve body 10 may be expandable, e.g., introduced in a collapsed configuration and, once positioned within the native annulus, expanded to dilate the surrounding tissue and to press the sewing cuff 20 against tissue around the native annulus without requiring sutures. For example, the valve body may include an expandable stent (not shown) carrying the leaflets, sewing cuff, and skirt, which may be introduced in a compressed state, e.g., using a transcatheter approach, and then deployed and expanded within the valve annulus.

Optionally, the skirt 30 may be manipulated as desired to facilitate securing the sewing cuff 20. For example, in one example, the skirt 30 may be provided in a folded, rolled, or other compressed state against the valve body 10 to facilitate observing and/or accessing the sewing cuff 20. For example, the skirt 30 in a compressed state, the sewing cuff 20 may be readily observable from the inlet end 12 of the valve body 1o to facilitate positioning and/or delivering sutures or other fasteners through the sewing cuff 20 and the adjacent tissue to secure the valve 8 relative to the native annulus. Alternatively, the skirt 30 may be provided expanded but may be sufficiently flexible such that the outer edge 34 of the skirt 30 may manipulated to facilitate observing past the skirt 30.

If additional sealing is desired, the skirt 30 may be placed against tissue around the native annulus and secured, e.g., using one or more sutures, such that the skirt 30 is offset from the sewing cuff 20 around the native annulus. For example, if the surgeon determines that additional sealing is desired, the skirt 30 may placed against the tissue adjacent the inlet of the native annulus and one or more sutures or other fasteners may be delivered to secure the outer edge 34 of the skirt 30 and enhance sealing and provide a hemostasis suture line to eliminate any potential paravalvular leaks around the valve 8.

Alternatively, if the surgeon determines that the sewing cuff 20 provides sufficient sealing, the skirt 30 may be removed, e.g., by separating the skirt 30 from the valve body 10 and removed from the patient's body. For example, as described above with respect to FIG. 1A, the suture 40 may be cut or untied, and a loose end pulled to unthread the suture 40 around the valve body 10 to released the skirt 30 and allow the skirt 30 to be removed. The procedure may then be completed using conventional methods.

Alternatively, with the configuration shown in FIGS. 2A and 2B, the valve body 10 may be positioned within the native annulus of an aortic valve with the outlet 14 adjacent or within the aortic root and the inlet adjacent the left ventricle (not shown). In this alternative, the skirt 30 may be positioned within the aortic root. Once the sewing cuff 20 is sutured or otherwise secured within the native annulus, the surgeon may determine whether additional sealing is desired and, if so, deliver one or more sutures to secure the skirt 30 to tissue around the outlet of the native annulus. Otherwise, if the valve 8 is sufficiently sealed by the sewing cuff 20, the skirt 30 may be removed as described above.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A prosthetic valve, comprising:
an annular valve body including an inlet end, an outlet end, and one or more valve members for selectively opening and closing a passage between the inlet and outlet ends;
a sewing cuff attached around an outer perimeter of the valve body; and
a flexible skirt removably attached around the outer perimeter of the valve body adjacent the sewing cuff.

2. The prosthetic valve of claim 1, wherein an inner edge of the skirt is attached around the outer perimeter by one or more sutures, the one or more sutures allowing the skirt to be separated from the valve body.

3. The prosthetic valve of claim 2, wherein the one or more sutures comprise a suture threaded in and out of the skirt material around the inner edge such that the suture may be cut to create a loose end that may be pulled to unthread or remove the suture from around the inner edge and release skirt from the valve body.

4. The prosthetic valve of claim 2, wherein the one or more sutures comprise a suture threaded in and out of the skirt material around the inner edge with opposite ends of the suture tied together by a knot such that the knot may be untied to allow one of the opposite ends to be pulled to unthread the suture from around the inner edge and release skirt from the valve body.

5. The prosthetic valve of claim 1, wherein the skirt comprises one or both of fabric or polyester.

6. (canceled)

7. The prosthetic valve of claim 1, wherein the skirt comprises a collapsed configuration to minimize interference with visualizing and/or securing the sewing cuff to a native valve annulus, the skirt expandable to contact tissue adjacent the native valve annulus to allow the skirt to be secured to tissue adjacent the native valve annulus.

8. The prosthetic valve of claim 1, wherein the skirt extends radially outwardly from the valve body.

9. The prosthetic valve of claim 8, wherein the skirt extends diagonally outwardly from the valve body.

10. The prosthetic valve of claim 8, wherein an outer edge of the skirt has a substantially uniform diameter.

11. The prosthetic valve of claim 8, wherein the skirt has a length between the inner edge and an outer edge of the skirt that is variable around the outer perimeter.

12. The prosthetic valve of claim 8, wherein skirt defines a plane extending radially outwardly from the valve body to an outer edge of the skirt.

13. The prosthetic valve of claim 12, wherein an outer edge of the skirt has a substantially uniform diameter.

14. The prosthetic valve of claim 12, wherein the skirt has a length between the inner edge and an outer edge of the skirt that is variable around the outer perimeter.

15. The prosthetic valve of claim 1, wherein the sewing cuff is attached around the outer perimeter at an intermediate location between the inlet and outlet ends, and the inner edge of the skirt is offset from the sewing cuff closer to one of the inlet and outlet ends.

16. The prosthetic valve of claim 15, wherein the inner edge of the skirt is secured adjacent the inlet end of the valve body.

17. The prosthetic valve of claim 15, wherein the inner edge of the skirt is secured adjacent the outlet end of the valve body.

18. The prosthetic valve of claim 1, wherein the valve body comprises an annulus from a tissue xenograft and the one or more valve members comprise tissue leaflets of the xenograft.

19. The prosthetic valve of claim 1, wherein the valve body comprises a non-tissue frame carrying the one or more valve members.

20. The prosthetic valve of claim 17, wherein the valve body comprises a mechanical valve.

21. A method for implanting a prosthetic valve comprising an annular valve body including an inlet end, an outlet end, and one or more valve members for selectively opening and closing a passage between the inlet and outlet ends, a sewing cuff attached around an outer perimeter of the valve body, and a flexible skirt removably attached around the outer perimeter of the valve body adjacent the sewing cuff, the method comprising:
introducing the prosthetic valve into a patient's body adjacent a valve annulus;
securing the sewing cuff around a perimeter of the valve annulus; and
either:
a) securing the skirt to tissue adjacent the valve annulus to further seal the prosthetic valve around the valve annulus; or
b) removing the skirt from the valve body.

22-30. (canceled)
